(19) United States
(12) Patent Application Publication
(10) Pub. No.: US 2026/0238225 A1
(43) Pub. Date: Aug. 13, 2026

(54) NOISE-SHAPING SAR-NESTED DSM FOR WIDE INPUT SIGNAL RANGE AND INPUT IMPEDANCE BOOSTING FOR BIO-SIGNAL RECORDING

(71) Applicant: Korea Advanced Institute of Science and Technology, Daejeon (KR)

(72) Inventors: Minkyu JE, Daejeon (KR); Kyeongwon JEONG, Daejeon (KR); Gichan YUN, Daejeon (KR); Sein OH, Daejeon (KR); Yoon Tae JUNG, Daejeon (KR); Dongyoun LEE, Daejeon (KR)

(73) Assignee: Korea Advanced Institute of Science and Technology, Daejeon (KR)

(21) Appl. No.: 19/639,465

(22) Filed: Apr. 6, 2026

Related U.S. Application Data
(63) Continuation of application No. PCT/KR2023/020438, filed on Dec. 12, 2023.

(30) Foreign Application Priority Data
Oct. 4, 2023 (KR) 10-2023-0131853

(51) Int. Cl. H03M 3/00

(52) U.S. Cl. CPC H03M 3/502

(57) ABSTRACT

A NS-SAR-nested DSM for a wide input signal range and input impedance boosting for bio-signal recording, and an operating method thereof, are provided. The DSM includes a CDS integrator configured to increase a CMRR and reduce noise; an input sampling capacitor configured to pre-sample a previous output of the DSM when the output is reflected to an input terminal to boost input impedance without an additional amplifier; an inverter-based amplifier configured to amplify an input signal to obtain a wide input signal range; an NS-SAR configured to increase resolution; and a truncation logic configured to, when output bits are reduced to predetermined bits and fed back to an input DAC, perform NS through the NS-SAR to reduce a truncation error and then perform truncation to feed back the truncated bits.

### CROSS-REFERENCE TO RELATED APPLICATIONS

This U.S. non-provisional application is a continuation application of PCT International Application PCT/KR2023/020438, which has an international filing date of December 12, 2023, and claims priorities under 35 U.S.C. 119 to Korean Patent Application No. 10-2023-0131853, filed on October 4, 2023, in the Korean intellectual property office, the disclosures of which are herein incorporated by reference in its entirety.

### BACKGROUND

### 1. Field of the Invention

The present disclosure relates to a noise-shaping (NS) successive approximation register (SAR)-nested delta-sigma modulator (DSM) for a wide input signal range and input impedance boosting for bio-signal recording, and an operating method thereof.

### 2. Description of the Related Art

Recently, there has been a growing interest in implantable and wearable devices for monitoring bio-signals. A number of implantable neural interface devices have been developed for the treatment of patients suffering from intractable diseases. In addition, wearable devices are rapidly being popularized and utilized in various healthcare applications, such as drowsiness detection and stress detection using bio-signals from wearable in-ear devices. By monitoring representative bio-signals such as an electroencephalogram (EEG), an electrocardiogram (ECG), an electromyogram (EMG), and a local field potential (LFP), the following therapeutic effects and advantages can be obtained.

EEG monitoring can assist in analyzing epileptic seizures and brain disorders, and ECG signal monitoring has been proven to provide valuable information regarding cardiovascular diseases. Similarly, muscle abnormalities can be detected by monitoring EMG signals. LFP signals can be effectively used in the treatment of neurological diseases such as Parkinson's disease and epilepsy.

To properly acquire such bio-signals, a recording front-end circuit must have the following characteristics: 1) low noise, 2) low power, 3) high input impedance, and 4) a wide dynamic range (DR). Above all, a recording integrated circuit (IC) must have sufficiently low noise because the amplitude of the signal is very small, ranging from several microvolts to several tens of millivolts. At the same time, the recording IC must consume low power to ensure the long-term operation of implantable and wearable devices, as most devices are driven by small batteries.

In addition, the input impedance must be sufficiently high for stable bio-signal recording. In general, since electrodes are required to measure bio-signals, electrode impedance must be considered. The electrode impedance is about 50 kΩ to 1 MΩ at a frequency of 1 kHz. Furthermore, when there is a mismatch between the electrode impedances, common-mode interference is converted into differential-mode interference, thereby interfering with bio-signal recording. To minimize input signal attenuation and the conversion of interference occurring at the electrode-tissue interface from a common mode to a differential mode, the input impedance of a bio-signal acquisition IC must be sufficiently high.

Lastly, DR is also an important factor in bio-signal acquisition circuits. This is because common-mode artifacts are a significant issue in wearable and implantable devices. Common causes of common-mode artifacts include the motion of a wearer of a wearable device and the electrical neural stimulation of a neural implant. Such common-mode artifacts are easily coupled to and saturate the recording channel, preventing the recording channel from continuously recording bio-signals. For example, in the case of a closed-loop implantable system, unwanted stimulation artifacts have the potential to interfere with and saturate the recording channel. In a wearable device, motions such as head movements and hand waving generate artifacts in the common-mode voltage of the signal, thereby interfering with signal acquisition in the recording channel. These artifacts can be as large as 100 mVPP and can easily distort the signal during the amplification stage, which typically has a large amplification gain. Consequently, there is a high probability that the artifacts will saturate the entire recording channel, and it may require a latency of 100 ms or more to recover back to normal operation.

To prevent these problems, a wide DR and a high common-mode rejection ratio (CMRR) are required. This ensures stable and continuous recording of wearable and implantable devices even under large-scale common-mode artifacts caused by user movement or simultaneous electrical stimulation.

### SUMMARY

An object of the present disclosure is to provide a DSM equipped with NS-SAR for high power efficiency and high resolution of an IC. Furthermore, an object of the present disclosure is to provide a technique for implementing fourth-order NS by providing a first stage integrator of the DSM configured to support not only a wide input swing but also high input impedance at low power consumption, an input sampling capacitor for improving the input impedance through a pre-sampling based charge transfer reduction technique that does not require an additional amplifier, and effectively shaping a truncation error into a noise shape without using an additional loop by feeding the truncation error back to a local NS-SAR loop.

In one aspect, an NS-SAR-nested DSM for a wide input signal range and input impedance boosting for bio-signal recording proposed in the present disclosure includes a correlated double sampling (CDS) integrator configured to increase a CMRR and reduce noise. The DSM further includes an input sampling capacitor configured to pre-sample a previous output of the DSM when the output of the DSM is reflected to an input terminal to boost input impedance without an additional amplifier. Moreover, the DSM includes an inverter-based amplifier configured to amplify an input signal to obtain a wide input signal range, an NS-SAR configured to increase resolution, and a truncation logic configured to, when output bits of the DSM are reduced to predetermined bits and fed back to and reflected in an input digital-to-analog converter (DAC), perform NS through the NS-SAR to reduce a truncation error and then perform truncation to feed back the truncated output bits to the input DAC.

In the input sampling capacitor, during an integration phase, the previous output of the DSM is integrated into the input sampling capacitor, and a charge difference between a current input voltage and the previous output is integrated into a feedback capacitor. During a pre-sampling phase, the previous output is pre-sampled in the input sampling capacitor by an asynchronous SAR analog-to-digital converter (ADC). During an input sampling phase, a next input voltage is newly charged in the input sampling capacitor.

The amplifier uses a current-reuse inverter-based amplifier that increases a linear input range of the amplifier by avoiding the use of a cascode stack for improved transconductance (GM), low power consumption, and a wide bandwidth.

The truncation logic decreases the output bits of the DSM to the predetermined bits and feeds them back to the input DAC, thereby feeding back the truncation error and reusing the input DAC to perform the NS again with low power consumption.

The NS-SAR uses a second-order NS-SAR, and the truncation error fed back to the input DAC through the truncation logic additionally passes through a second-order NS-SAR noise transfer function (NTF) so that second-order NS is additionally generated, thereby performing a total of fourth-order NS.

In another aspect, a method of operating an NS-SAR-nested DSM for a wide input signal range and input impedance boosting for bio-signal recording proposed in the present disclosure includes increasing a CMRR and reducing noise through a CDS integrator. The method further includes pre-sampling a previous output of the DSM into an input sampling capacitor when an output of the DSM is reflected to an input terminal to boost input impedance without an additional amplifier, and amplifying an input signal using an inverter-based amplifier to obtain a wide input signal range. Furthermore, the method includes increasing resolution through a NS-SAR. Finally, the method includes, when output bits of the DSM are reduced to predetermined bits and fed back to and reflected in an input DAC, performing NS through the NS-SAR to reduce a truncation error, and then performing truncation through a truncation logic to feed back the truncated output bits to the input DAC.

According to embodiments of the present disclosure, an integrator of the NS-SAR-nested DSM for a wide input signal range and input impedance boosting for bio-signal recording supports a wide input swing as well as a high input impedance at low power consumption. Furthermore, the input impedance may be further improved through a pre-sampling based charge transfer reduction technique that does not require an additional amplifier, and charge transfer may be reduced to increase the input impedance by pre-charging a sampling capacitor using only a previously sampled signal. In addition, by employing a novel truncation error shaping method according to an embodiment of the present disclosure, a truncation error is fed back to a local NS-SAR loop, such that the truncation error is effectively noise-shaped without utilizing an additional loop, thereby generating fourth-order NS.

### BRIEF DESCRIPTION OF THE DRAWINGS

FIG. 1 is a diagram illustrating an NS-SAR-nested DSM for a wide input signal range and input impedance boosting for bio-signal recording according to an embodiment of the present disclosure.

FIG. 2 is a flowchart illustrating a method of operating the NS-SAR-nested DSM according to an embodiment of the present disclosure.

FIG. 3A is a diagram illustrating a case where an independent capacitor is used when an output of a DSM according to the related art is reflected to an input terminal.

FIG. 3B is a diagram illustrating an input sampling capacitor according to an embodiment of the present disclosure.

FIG. 4A is a diagram illustrating an integration phase for explaining a process of pre-sampling a previous output of a DSM into an input sampling capacitor according to an embodiment of the present disclosure.

FIG. 4B is a diagram illustrating a pre-sampling phase for explaining the process of pre-sampling the previous output of the DSM into the input sampling capacitor according to an embodiment of the present disclosure.

FIG. 4C is a diagram illustrating an input sampling phase for explaining the process of pre-sampling the previous output of the DSM into the input sampling capacitor according to an embodiment of the present disclosure.

FIG. 5 is a diagram illustrating an amplifier according to an embodiment of the present disclosure.

FIG. 6 is a diagram for explaining an amplifier error according to an embodiment of the present disclosure.

FIG. 7A is a graph illustrating power spectral density (PSD) with respect to a normalized frequency to explain a noise level according to an embodiment of the present disclosure.

FIG. 7B is a graph illustrating PSD with respect to a frequency to explain the noise level according to an embodiment of the present disclosure.

FIG. 8A is a diagram illustrating a truncation logic to explain a truncation error of a DAC according to an embodiment of the present disclosure.

FIG. 8B is a diagram illustrating a phase of reflecting a truncated result back to a capacitor DAC of a SAR according to an embodiment of the present disclosure.

FIG. 8C is a diagram illustrating a phase of removing a truncation error using a NS loop according to an embodiment of the present disclosure.

FIG. 9 is a graph showing measured Signal-to-noise and distortion ratio (SNDR) and DR performance according to an embodiment of the present disclosure.

FIG. 10 is a graph showing truncation error results depending on the presence or absence of a truncation logic according to an embodiment of the present disclosure.

FIG. 11 is a graph showing input-referred noise (IRN) performance according to an embodiment of the present disclosure.

FIG. 12 is a diagram illustrating measured CMRR and input impedance according to an embodiment of the present disclosure.

FIG. 13A is a chip micrograph according to an embodiment of the present disclosure.

FIG. 13B is a graph showing ECG recording results according to an embodiment of the present disclosure.

### DETAILED DESCRIPTION

The present disclosure proposes a NS-SAR-nested DSM for a wide input signal range and input impedance boosting for bio-signal recording. An IC according to an embodiment of the present disclosure directly converts a small input signal into a digital signal based on a first-order DSM equipped with a second-order NS-SAR for high power efficiency and high resolution.

A first-stage integrator of the DSM according to an embodiment of the present disclosure may be designed to support a wide input swing as well as a high input impedance at low power consumption. In addition, the input impedance may be further improved through a proposed pre-sampling-based charge transfer reduction technique that does not require an additional amplifier. Furthermore, charge transfer may be reduced and the input impedance may be increased by pre-charging a sampling capacitor using only a previously sampled signal.

A novel truncation error shaping method according to an embodiment of the present disclosure is proposed. By feeding the truncation error back to a local NS-SAR loop, the truncation error may be effectively noise-shaped without using an additional loop, thereby generating fourth-order NS. Hereinafter, embodiments of the present disclosure will be described in detail with reference to the accompanying drawings.

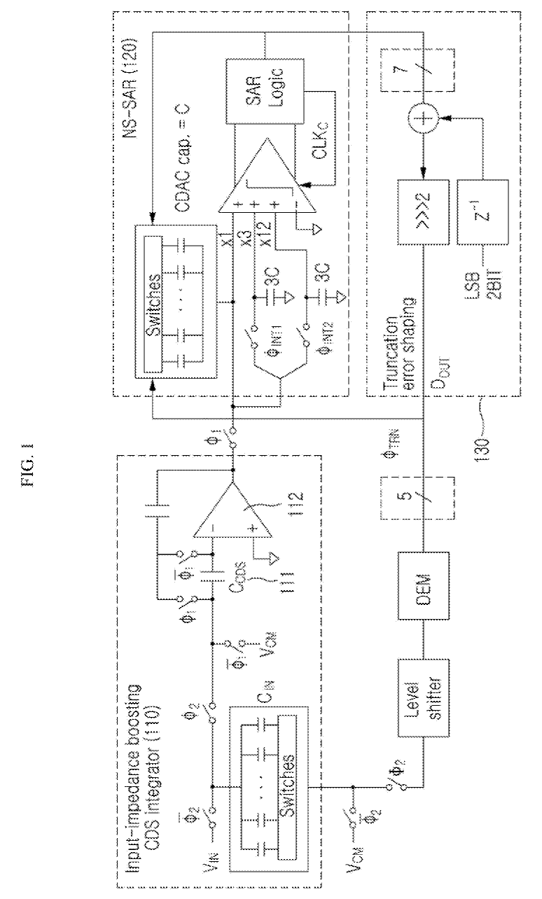

FIG. 1 is a diagram illustrating a NS-SAR-nested DSM for a wide input signal range and input impedance boosting for bio-signal recording according to an embodiment of the present disclosure. The DSM according to an embodiment of the present disclosure includes a CDS integrator 110, an input sampling capacitor 111, an amplifier 112, an NS-SAR 120, and a truncation logic 130.

The CDS integrator 110 according to an embodiment of the present disclosure may increase a CMRR and reduce noise. The input sampling capacitor 111 according to an embodiment of the present disclosure pre-samples a previous output of the DSM when the output of the DSM is reflected to an input terminal to boost the input impedance without an additional amplifier. The amplifier 112 according to an embodiment of the present disclosure amplifies a signal on an inverter basis to obtain a wide input signal range. The NS-SAR 120 according to an embodiment of the present disclosure may increase resolution. When output bits of the DSM are reduced to predetermined bits and fed back to and reflected in an input DAC, the truncation logic 130 according to an embodiment of the present disclosure performs NS through the NS-SAR to reduce a truncation error and then performs truncation to feed back the truncated output bits to the input DAC.

There is a trade-off between 1/f noise reduction and input impedance, which is mainly due to the use of chopper stabilization. To overcome this trade-off, an embodiment of the present disclosure proposes an NS-SAR-nested DSM structure that can reduce 1/f noise and simultaneously boost input impedance without using an additional amplifier.

Referring to FIG. 1, a block diagram of the proposed DSM is illustrated, which consists of a first-order DSM and a second-order NS-SAR. High resolution is implemented with low power consumption by using an energy-efficient NS-SAR ADC rather than a structure using an amplifier-based multi-stage integrator. In addition, an input impedance boosting method capable of obtaining 1/f noise reduction and boosted input impedance by minimizing charge transfer at the input of the CDS integrator 110 is proposed. For the input impedance boosting, the proposed pre-sampling-based charge transfer reduction technique that minimizes charge transfer to the input capacitor 111 in a discrete-time DSM (DT-DSM) is used. Due to the proposed method, an additional amplifier for input impedance boosting is not required. The CDS integrator 110 is also optimized to achieve a wide linear input range with high power efficiency. Finally, a novel truncation error shaping technique reusing the NS-SAR is proposed.

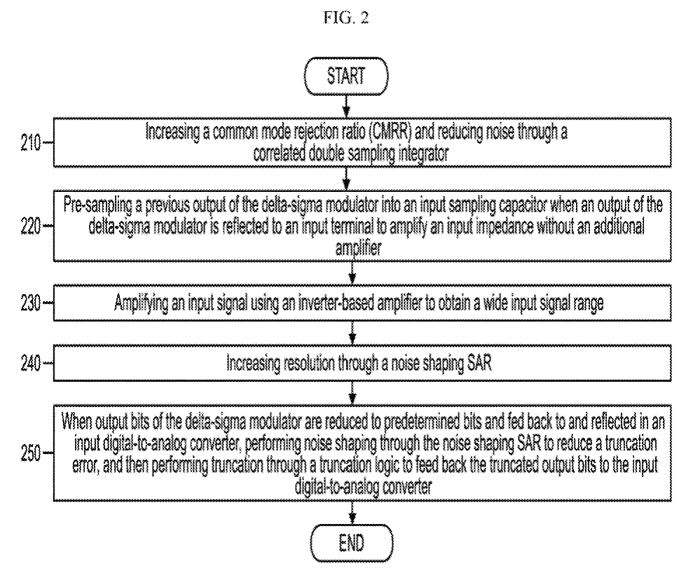

FIG. 2 is a flowchart illustrating an operating method of the NS-SAR-nested DSM for a wide input signal range and input impedance boosting for bio-signal recording according to an embodiment of the present disclosure.

The operating method of the NS-SAR-nested DSM for a wide input signal range and input impedance boosting for bio-signal recording according to an embodiment of the present disclosure includes increasing a CMRR and reducing noise through a CDS integrator (step 210). The method includes pre-sampling a previous output of the DSM into an input sampling capacitor when the output of the DSM is reflected to an input terminal to boost an input impedance without an additional amplifier (step 220). The method includes amplifying an input signal using an inverter-based amplifier to obtain a wide input signal range (step 230). The method includes increasing resolution through a NS-SAR (step 240). Finally, the method includes, when output bits of the DSM are reduced to predetermined bits and fed back to and reflected in an input DAC, performing NS through the NS-SAR to reduce a truncation error, and then performing truncation through a truncation logic to feed back the truncated output bits to the input DAC (step 250).

According to an embodiment of the present disclosure, in an integration phase, the previous output of the DSM is integrated into the input sampling capacitor, and a charge difference between a current input voltage and the previous output is integrated into a feedback capacitor. In a pre-sampling phase according to an embodiment of the present disclosure, pre-sampling is performed on the input sampling capacitor by an asynchronous SAR ADC. In an input sampling phase according to an embodiment of the present disclosure, a next input voltage is newly charged to the input sampling capacitor.

According to an embodiment of the present disclosure, a current-reuse inverter-based amplifier is used, which increases a linear input range of the amplifier by not using a cascode stack for improved GM, low power, and a wide bandwidth.

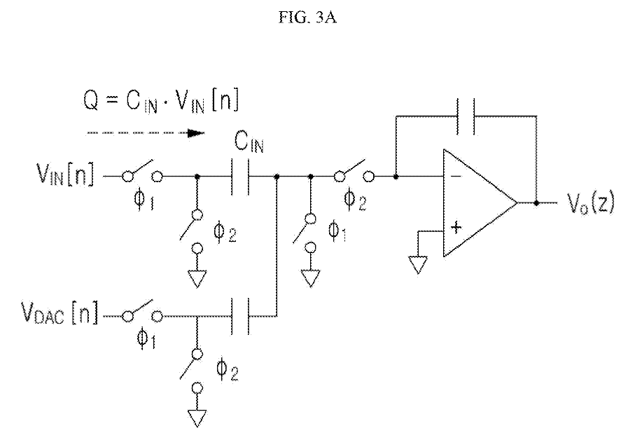

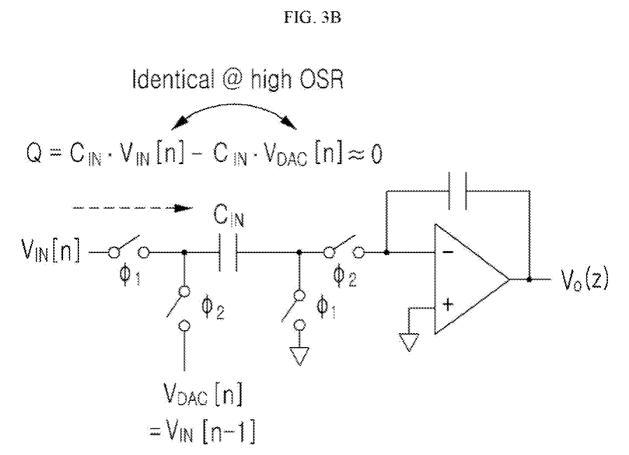

According to an embodiment of the present disclosure, the output bits of the DSM are reduced to the predetermined bits and fed back to the input DAC, thereby feeding back the truncation error and reusing the input DAC to perform the NS again with low power consumption. Furthermore, according to an embodiment of the present disclosure, a second-order NS-SAR is used, and the truncation error fed back to the input DAC through the truncation logic additionally passes through a second-order NS-SAR NTF, thereby additionally generating second-order NS, such that a total of fourth-order NS may be performed. Hereinafter, with reference to FIGS. 3 to 8, an operation process of the NS-SAR-nested DSM for a wide input signal range and input impedance boosting for bio-signal recording according to an embodiment of the present disclosure will be described in more detail.

FIG. 3A is a diagram illustrating a case where an independent capacitor is used when an output of a DSM according to the related art is reflected to an input terminal.

FIG. 3B is a diagram illustrating an input sampling capacitor according to an embodiment of the present disclosure.

The input sampling capacitor (CIN in FIG. 3B) according to an embodiment of the present disclosure pre-samples a previous output of the DSM when the output of the DSM is reflected to the input terminal to boost an input impedance without an additional amplifier. When the previous output of the DSM is pre-sampled into the capacitor that sampled a current input, and then a new input is sampled, charge transfer at the input is minimized. This becomes more advantageous as a sampling frequency increases.

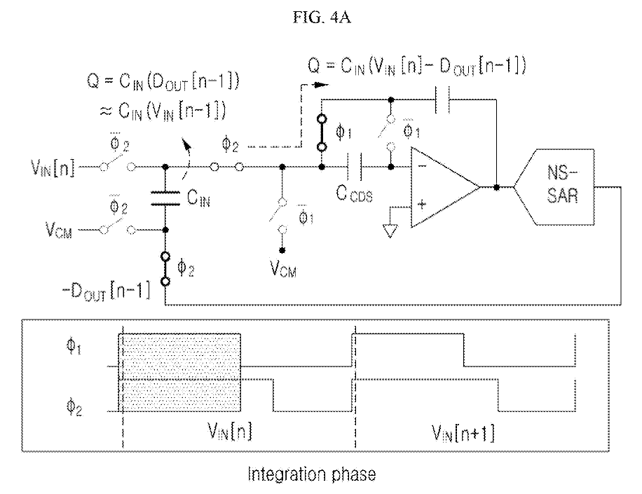

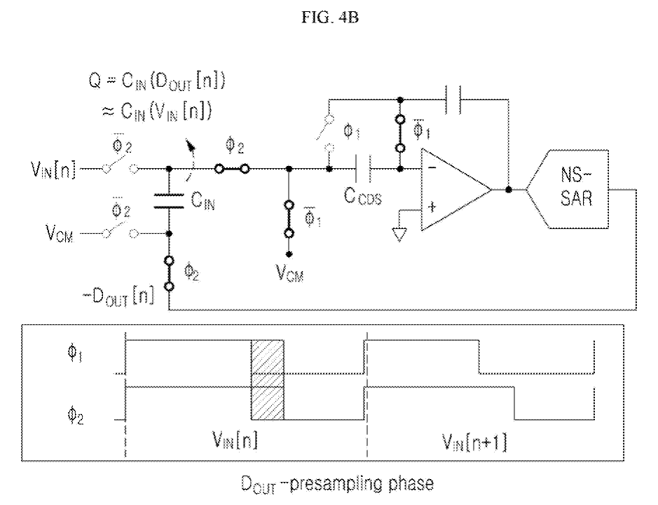

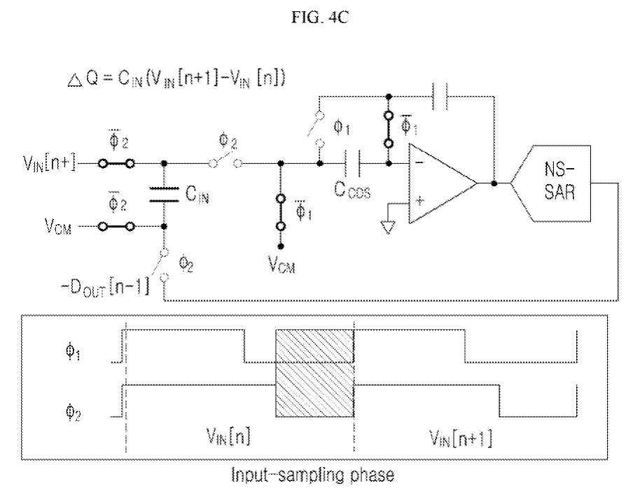

FIG. 4A is a diagram illustrating an integration phase for explaining a process of pre-sampling a previous output of a DSM into an input sampling capacitor according to an embodiment of the present disclosure.

FIG. 4B is a diagram illustrating a pre-sampling phase for explaining the process of pre-sampling the previous output of the DSM into the input sampling capacitor according to an embodiment of the present disclosure.

FIG. 4C is a diagram illustrating an input sampling phase for explaining the process of pre-sampling the previous output of the DSM into the input sampling capacitor according to an embodiment of the present disclosure.

To acquire a bio-signal regardless of electrode impedance or mismatch, high input impedance characteristics are essential in a bio-signal recording circuit.

In the proposed structure, both input signal sampling and DOUT feedback are performed at the same CIN of the CDS integrator. Therefore, charge transferred from the input to CIN during input sampling can be significantly reduced. To further reduce the amount of charge transferred to CIN to obtain a higher input impedance, a delay between input sampling and DOUT feedback must be minimized. As an oversampling ratio (OSR) increases, the amount of charge transfer decreases and the input impedance increases.

In the present disclosure, a pre-sampling-based charge transfer reduction technique is proposed to further improve an input impedance boosting effect. Circuit operation in the integration phase according to an embodiment of the present disclosure is the same as in the related art, as shown in FIG. 4A. In the integration phase, a D_{OUT}[n-1] result is applied to a bottom terminal of C_{IN}. At a high OSR, D_{OUT}[n-1] is close to Vin[n-1]. Then, a charge difference between C_{IN}Vin[n] and C_{IN}D_{OUT}[n-1] of the input capacitor is transferred to and integrated into a feedback capacitor. Unlike the previous work, the integration phase is followed by a new phase called a D_{OUT}-presampling phase. FIG. 4B shows the operation of the pre-sampling phase. During this phase, D_{OUT}[n] is generated within 100 ns by an asynchronous SAR ADC. Then, the updated D_{OUT}[n] is pre-sampled into C_{IN}. Finally, in the input sampling phase (FIG. 4C), Vin[n+1] is newly charged to C_{IN}. Therefore, a charge difference between C_{IN}Vin[n+1] and C_{IN}Vin[n] is transferred from the input to C_{IN}. Consequently, the pre-sampling-based charge transfer reduction technique proposed in the present disclosure involves only a 1-sample delay between the input and D_{OUT} feedback to C_{IN}, whereas a 2-sample delay occurs in the related art. Due to this improvement, the boosting effect can be doubled at the same OSR. Therefore, an overall structure of the proposed DT-DSM to which the present CDS integrator is applied reduces 1/f noise while improving the CMRR and the input impedance.

However, there are some challenges in implementing the proposed circuit structure. Additional power is consumed to pre-charge an input capacitive DAC (C-DAC) with a new D_{OUT}, while an additional amplifier is not required for input impedance boosting. To mitigate DT-DSM noise, a C-DAC size must be set relatively large. Due to these characteristics, power and area consumption may increase to some extent. In addition, utilizing the DT-DSM structure requires a strong reference buffer to quickly establish a reference voltage for the C-DAC and to prevent an input-dependent loading effect. Since the number of bits fed back to the input C-DAC affects the input impedance, there is a limit to truncating the number of feedback bits. Furthermore, the introduction of a pre-sampling time reduces the time to sample a new input. Since it becomes more difficult to increase the OSR, the DSM structure is designed as a third-order to achieve target resolution.

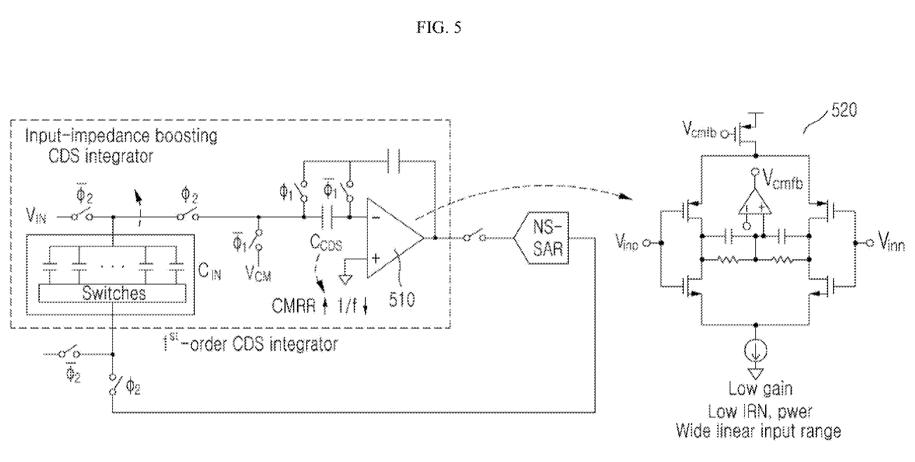

FIG. 5 is a diagram illustrating an amplifier according to an embodiment of the present disclosure. A discrete DSM according to an embodiment of the present disclosure has an advantage of being able to use a CDS integrator, but has poor power efficiency. This is because the bandwidth must also be increased while increasing the gain of the amplifier. Therefore, a small gain is used and a high bandwidth is maintained to reduce power consumption. According to an embodiment of the present disclosure, an inverter-based amplifier 510 is used in this process. If an internal structure 520 of the inverter-based amplifier according to an embodiment of the present disclosure is implemented with a low gain, there is no need to stack MOSFETs in a cascode manner, and as a result, it can have a wide input signal range of 600 mV_{PP}.

Since DSM quantization noise according to an embodiment of the present disclosure is shaped with third-order characteristics, it can be lowered to a level below the IRN of the DSM. However, a NTF of the DSM can be easily degraded depending on amplifier characteristics. Therefore, the amplifier must be optimized for excellent noise performance.

Referring to FIG. 5, an amplifier structure 520 according to an embodiment of the present disclosure is shown. A current-reuse inverter-based amplifier is used for improved G_{M}. However, since a bandwidth of the amplifier used in the integrator must be much wider than a sampling frequency, power consumption generally increases. Therefore, in the present disclosure, the amplifier is optimally designed to have a moderate gain of 36 dB so that a wide bandwidth can be implemented while consuming low power. In this design, a unit gain bandwidth of 480 kHz can be obtained by consuming a current of 2.2 µA. Furthermore, in the present disclosure, a linear input range of the amplifier is also achieved by not using a cascode stack for an unneeded high output resistance. For a low IRN, a size of an input transistor must be large enough, which causes a gate leakage problem. Therefore, a thick gate oxide transistor is used as the input transistor of the amplifier to reduce transistor gate leakage.

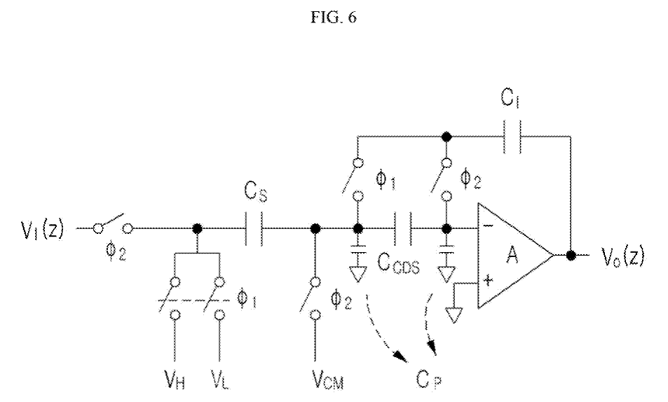

FIG. 6 is a diagram for explaining an amplifier error according to an embodiment of the present disclosure. However, a moderate amplifier gain may affect the NTF of the DSM. This is because as the amplifier gain decreases, the overall DSM loop gain decreases. Additionally, parasitic capacitance generated due to the large input transistor of the amplifier has the potential to degrade integrator performance. A finite gain and parasitic capacitance of the amplifier can lead to gain and phase errors, as shown in FIG. 6. According to an embodiment of the present disclosure, as the gain of the integrator decreases due to the inverter-based amplifier, a phase error and a gain error occur. Since the gain error can be adjusted using a SAR ADC reference voltage, it does not substantially have a significant effect on the DSM, but the phase error affects the overall DSM operation.

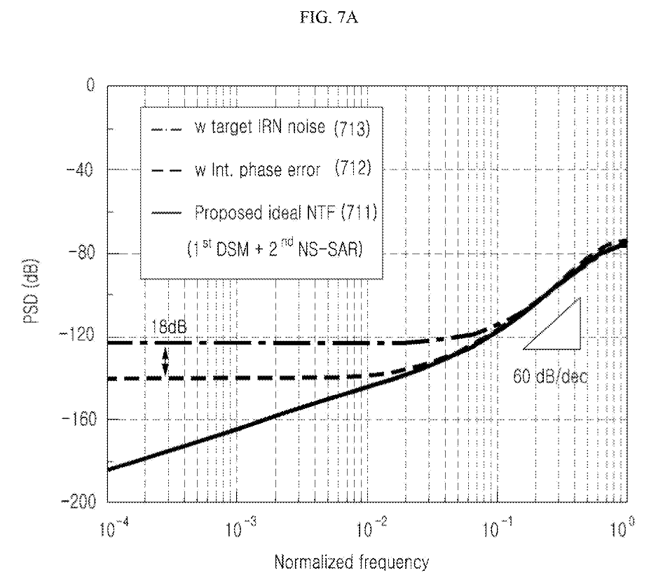

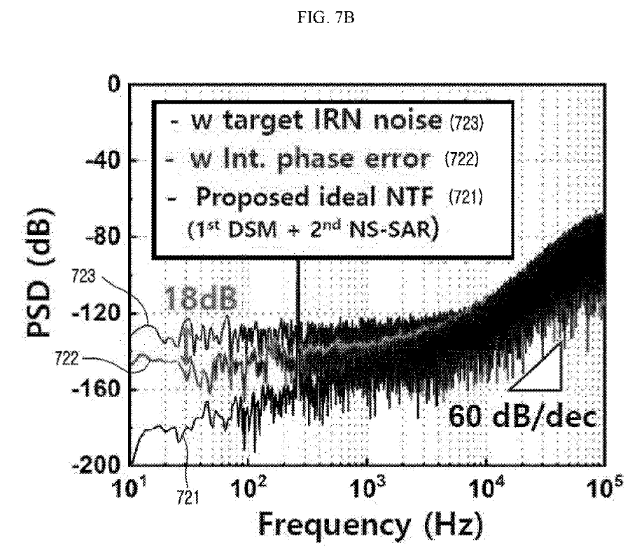

FIG. 7A is a graph illustrating PSD with respect to a normalized frequency to explain a noise level according to an embodiment of the present disclosure.

FIG. 7B is a graph illustrating PSD with respect to a frequency to explain the noise level according to an embodiment of the present disclosure.

By using a small gain in the integrator, characteristics deteriorate as if noise has increased (712, 722) compared to existing noise conversion formulas (711, 721). However, this is not a problem because target noise levels (731, 732) are higher. FIGS. 7A and 7B compare the IRN to show the DSM noise of the proposed DSM and the degradation due to a phase error. An ideal NTF of the proposed DSM is drawn with a black line 711 in FIG. 7A and a black line 721 in FIG. 7B. Quantization noise is significantly lowered by a combination of the first-order DSM and the second-order NS-SAR. However, in the embodiment of the present disclosure, the gain of the amplifier used in the integrator is limited to 36 dB, resulting in a phase error of 1.84%. Furthermore, the performance of the amplifier G_{M} may be degraded due to process variations. In a worst-case scenario where the gain is further reduced by 6 dB, a phase error of approximately 3% is assumed. As can be seen from a red line 712 in FIG. 7A and a red line 722 in FIG. 7B, NTF degradation occurs due to this phase error because a loop gain decreases at a low frequency. Although integrator performance is degraded due to the low amplifier gain and parasitic capacitance, a degraded noise level due to an integrator phase error is still much lower than the target noise level drawn by a blue line 731 in FIG. 7A and a blue line 732 in FIG. 7B.

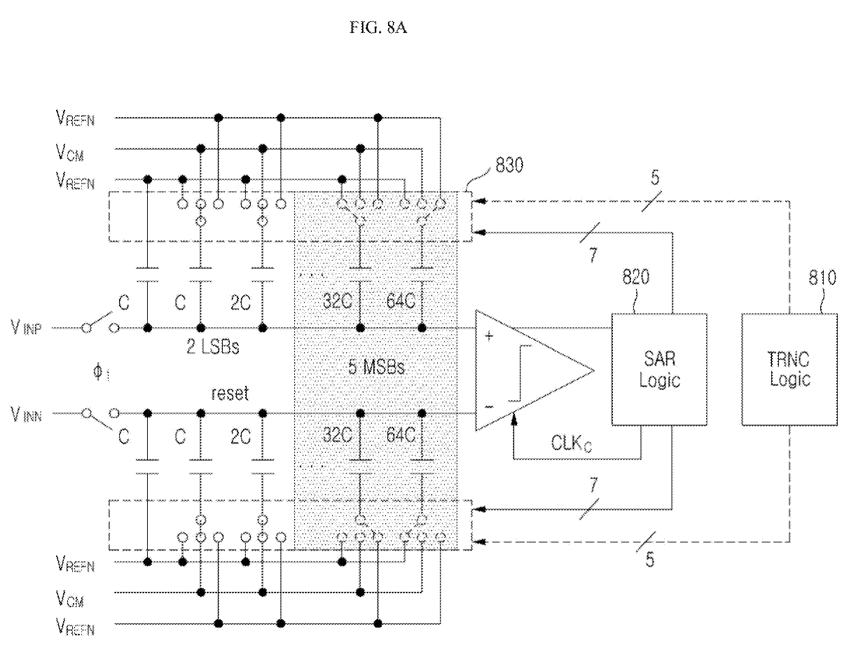

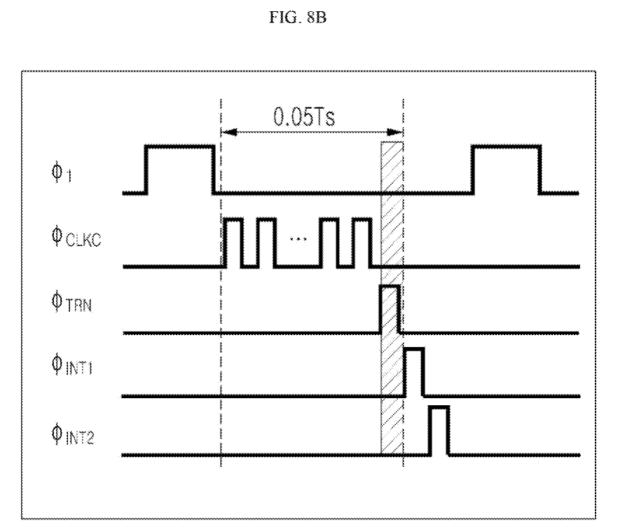

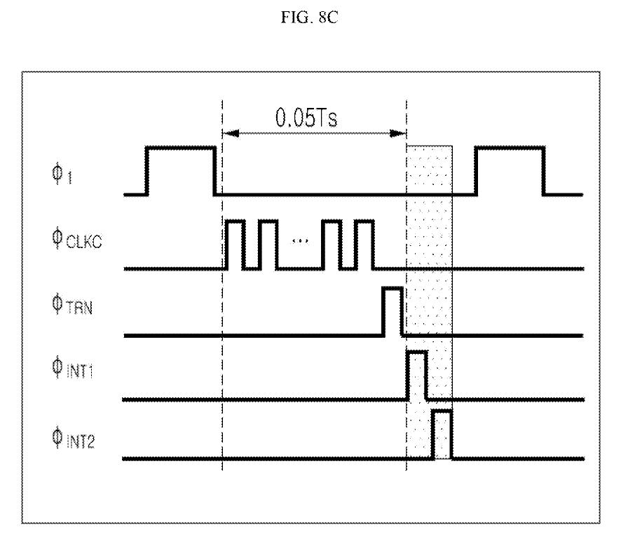

FIG. 8A is a diagram illustrating a truncation logic to explain a truncation error of a DAC according to an embodiment of the present disclosure.

FIG. 8B is a diagram illustrating a phase of reflecting a truncated result back to a capacitor DAC of a SAR according to an embodiment of the present disclosure.

FIG. 8C is a diagram illustrating a phase of removing a truncation error using a NS loop according to an embodiment of the present disclosure.

In a DSM, increasing the number of output bits improves performance. However, as the number of bits increases, a linearity problem occurs in an input DAC. Therefore, in an embodiment of the present disclosure, a 7-bit output is reduced to 5 bits and reflected as a feedback to the input DAC. A structure that discards bits in this manner is referred to as truncation, and a truncation error occurs during this process. In general, this is removed by additional NS, which consumes power in the process. In an embodiment of the present disclosure, the truncation error is removed by using an NS loop.

When an output of a NS-SAR 820 is generated in the circuit, a result truncated through a truncation logic 810 is reflected back to a C-DAC 830 of the SAR, so that the truncation error is also stored back in the C-DAC. Thereafter, by performing NS again, both the truncation error and a quantization error are simultaneously noise-shaped.

More specifically, according to an embodiment of the present disclosure, to obtain second-order NS without using an active amplifier structure, an asynchronous 7-bit NS-SAR ADC structure is used to quantize an integrator output in the proposed DSM. As shown in FIG. 8A, a passive second-order NS is utilized to achieve high resolution and low power consumption. Since a total capacitor size of the C-DAC is 1 pF, a passive integration capacitor may be determined as 3 pF for a passive integration coefficient of 1/(1-0.75z⁻¹).

In general, an NS-SAR ADC is vulnerable to low-frequency applications due to leakage current of switches. As a process scales down, the leakage problem becomes more severe. Therefore, the number of switches can have a significant impact on the performance of the NS-SAR. In low-frequency applications, even if a second-order or higher NS is performed using an NS-SAR, a second-order or higher NS cannot be obtained if many switches are used in a 65 nm process. As a result, the proposed NS-SAR scheme with the smallest number of switches is used, and thick oxide transistors are used for the NS switches due to the leakage problem. Furthermore, according to an embodiment of the present disclosure, the C-DAC may be implemented using custom-designed metal-oxide-metal (MOM) capacitors to minimize C-DAC mismatch. In an embodiment of the present disclosure, a 7-bit asynchronous SAR ADC with top-plate sampling is proposed.

In addition, a novel fourth-order truncation error shaping structure is proposed by reusing the NS-SAR ADC. An NS-SAR ADC can generally generate high resolution with low power consumption. Therefore, a passive second-order NS-SAR ADC was selected as a quantizer of the DSM. However, when a high-resolution ADC output is fed back to the input C-DAC of the DSM, a linearity problem occurs. In this case, a multi-bit ADC output is generally truncated, and a resulting truncation error is in the form of noise in a digital domain. Conventional truncation error shaping techniques generally select second-order shaping in consideration of the complexity and power consumption of digital logic. If the number of truncated bits increases beyond the second order, it becomes much more difficult to filter the truncation error, requiring a more complex circuit and higher power consumption. In the present disclosure, a novel truncation error shaping method is proposed, and noise can be further shaped with low power consumption by reusing the NS-SAR ADC.

A truncation error according to an embodiment of the present disclosure is fed back to the C-DAC 830 of the NS-SAR ADC. Therefore, the truncation error additionally passes through the NS SAR NTF. In this way, second-order NS is additionally generated due to the truncation error, resulting in a total of fourth-order NS. Here, it has a third-order NTF due to the characteristics of the first-order DSM and the second-order NS-SAR. However, to mitigate C-DAC complexity and linearity problems, if a 7-bit output is truncated to 5 bits before being fed back to the input C-DAC, noise performance is significantly degraded due to a truncation error. By applying the proposed technique, the NTF has fourth-order shaping while having the same in-band characteristics as the case of 7-bit feedback.

Referring to FIG. 8A, a circuit implementation of the proposed truncation error shaping is shown. After an asynchronous SAR 810 conversion, the truncated bits are supplied to the SAR C-DAC 830 and two LSBs are reset. Then, an active second-order NS is performed. As a result, a truncation error and quantization noise are shaped simultaneously. The truncated 5-bit output is a result of truncating the two LSBs by summing the 7-bit NS-SAR output and the previously truncated two LSBs. The truncation error is fed back to the C-DAC in the form of first-order noise in the digital domain. The processed 5 MSB result is applied to a logic circuit that switches a bottom plate of the C-DAC through an additional multiplexer as shown in FIG. 8B. When each bit conversion result is applied to the C-DAC, Φ_{TRN} rises from 0 to 1. After obtaining the truncated 5-bit result in the truncation logic of FIG. 8A, Φ_{TRN} becomes High and the truncated 5-bit result is applied to the C-DAC. The proposed truncation error shape may affect an entire input swing range. However, this only leads to a slight decrease in an output swing.

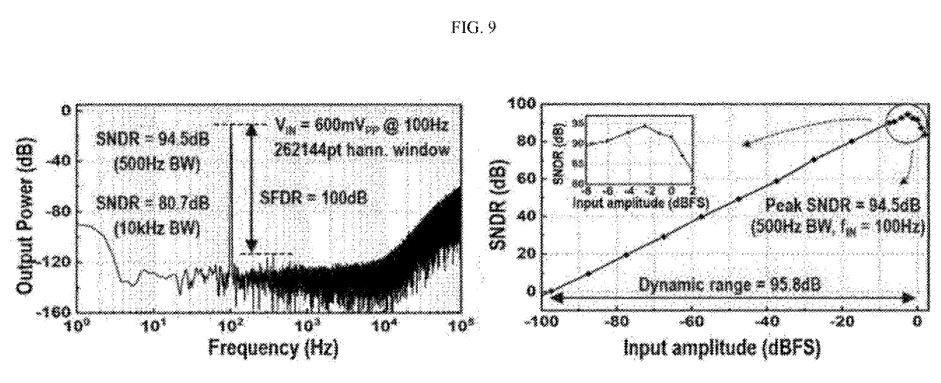

FIG. 9 is a graph showing measured SNDR and DR performance according to an embodiment of the present disclosure. An IC fabricated with 65 nm CMOS according to an embodiment of the present disclosure consumes 5.2 µW and 0.1292 mm2. When a 600 mV_{PP} sinusoidal input is applied, measured peak SNDRs are 94.5 dB and 80.7 dB for 500 Hz and 10 kHz BW, respectively, whereas a measured DR is 95.8 dB.

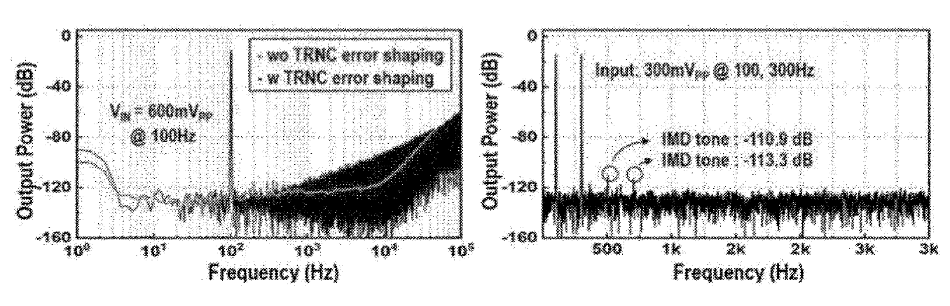

FIG. 10 is a graph showing truncation error results depending on the presence or absence of a truncation logic according to an embodiment of the present disclosure. It shows two-tone test results demonstrating TRNC errors and artifact tolerance depending on the presence or absence of the proposed truncation logic.

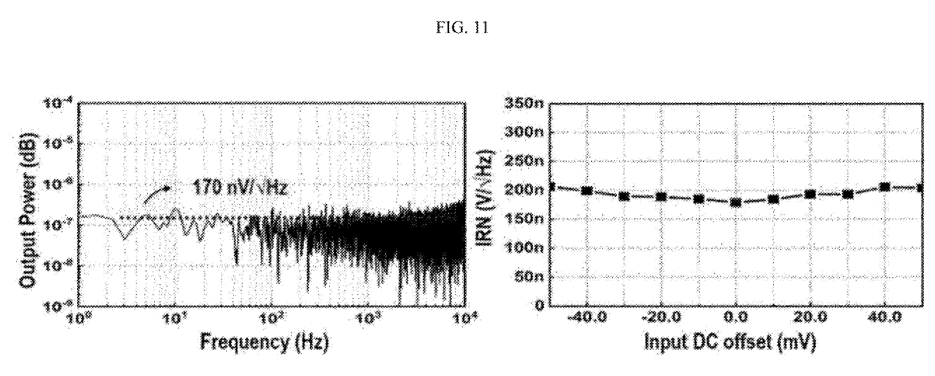

FIG. 11 is a graph showing IRN performance according to an embodiment of the present disclosure. A measured IRN is 174 nV/√Hz, and even if an input DC offset changes, the IRN does not change significantly.

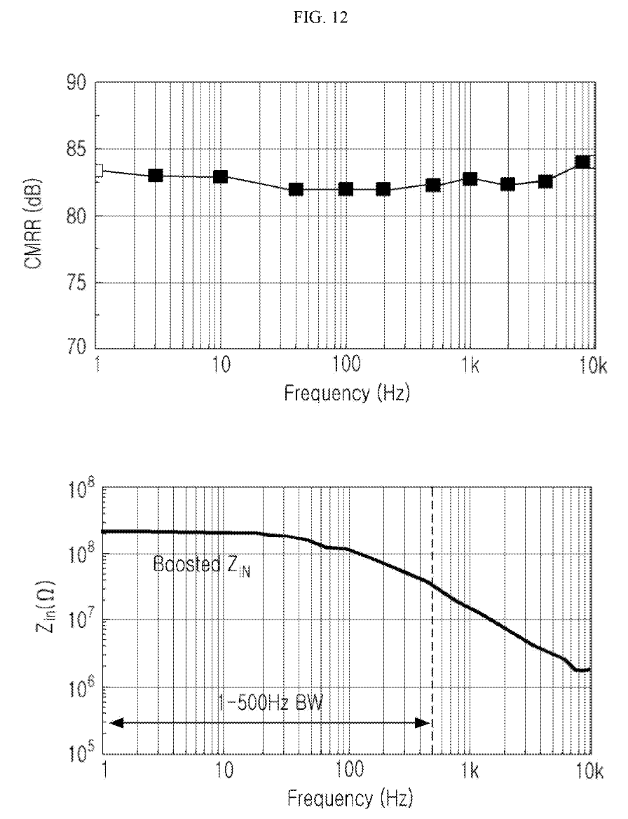

FIG. 12 is a diagram showing measured CMRR and input impedance according to an embodiment of the present disclosure. A measured CMRR is higher than 83 dB, and Z_{IN} is measured as 208 MΩ at DC and 31.5 MΩ at 500 Hz.

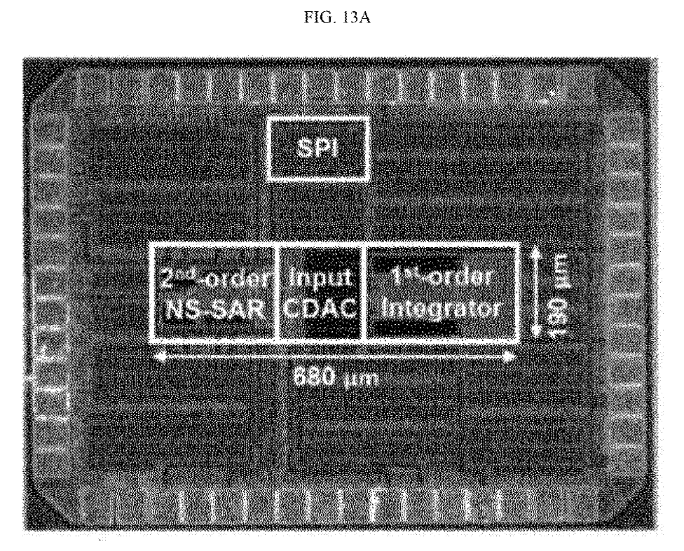

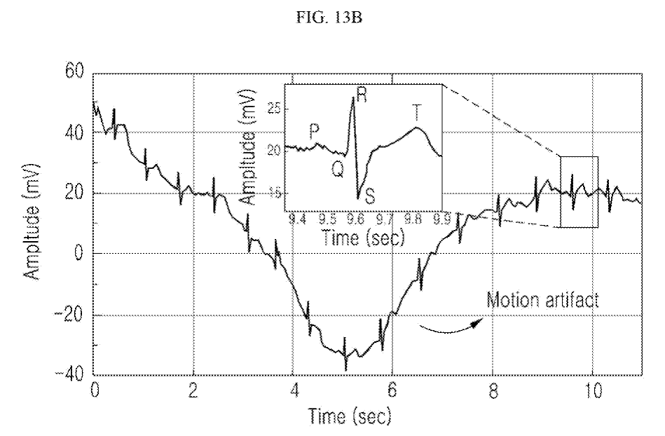

FIGS. 13A and 13B are diagrams showing a chip micrograph and ECG recording results according to an embodiment of the present disclosure. FIG. 13A shows a chip micrograph and FIG. 13B shows an ECG signal recorded using a fabricated IC, demonstrating that an ECG waveform is well separated despite a motion artifact. The design according to an embodiment of the present disclosure achieves a wider linear input range and excellent SNDR and DR performance. Due to CDS, a high CMRR and low 1/f noise can be obtained. In addition, Z_{IN} can be boosted without using a separate amplifier. The proposed IC achieves near-state-of-the-art 174.3 dB FOMSNDR and 175.6 dB FOMDR.

Although the embodiments have been described with reference to the limited embodiments and drawings, various modifications and variations can be made by those skilled in the art from the above description. For example, suitable results may be achieved if the described techniques are performed in a different order from the described method, and/or components of the described system, structure, device, circuit, etc., are combined or integrated in a different form from the described method, or replaced or substituted by other components or equivalents. Therefore, other implementations, other embodiments, and equivalents to the claims also fall within the scope of the appended claims.

What is claimed is:

1. A DSM comprising: a CDS integrator configured to increase a CMRR and reduce noise; an input sampling capacitor configured to pre-sample a previous output of the DSM when an output of the DSM is reflected to an input terminal to boost an input impedance without an additional amplifier; an inverter-based amplifier configured to amplify an input signal to obtain a wide input signal range; an NS-SAR configured to increase resolution; and a truncation logic configured to, when output bits of the DSM are reduced to predetermined bits and fed back to and reflected in an input DAC, perform NS through the NS-SAR to reduce a truncation error and then perform truncation to feed back the truncated output bits to the input DAC.

2. The DSM of claim 1, wherein the input sampling capacitor is configured such that: in an integration phase, the previous output of the DSM is integrated into the input sampling capacitor, and a charge difference between a current input voltage and the previous output is integrated into a feedback capacitor; in a pre-sampling phase, the previous output is pre-sampled in the input sampling capacitor by an asynchronous SAR ADC; and in an input sampling phase, a next input voltage is newly charged in the input sampling capacitor.

3. The DSM of claim 1, wherein the amplifier uses a current-reuse inverter-based amplifier configured to increase a linear input range of the amplifier by not using a cascode stack for improved G_{M} low power consumption, and a wide bandwidth.

4. The DSM of claim 1, wherein the truncation logic is configured to reduce the output bits of the DSM to the predetermined bits and feed the reduced bits back to the input DAC, thereby feeding back the truncation error and reusing the input DAC to perform the NS again with low power consumption.

5. The DSM of claim 4, wherein the NS-SAR uses a second-order NS-SAR, and the truncation error fed back to the input DAC through the truncation logic additionally passes through a second-order NS-SAR NTF so that second-order NS is additionally generated, thereby performing a total of fourth-order NS.

6. A method of operating a DSM, the method comprising: increasing a CMRR and reducing noise through a CDS integrator; pre-sampling a previous output of the DSM into an input sampling capacitor when an output of the DSM is reflected to an input terminal to boost an input impedance without an additional amplifier; amplifying an input signal using an inverter-based amplifier to obtain a wide input signal range; increasing resolution through a NS-SAR; and when output bits of the DSM are reduced to predetermined bits and fed back to and reflected in an input DAC, performing NS through the NS-SAR to reduce a truncation error, and then performing truncation through a truncation logic to feed back the truncated output bits to the input DAC.

7. The method of claim 6, wherein the pre-sampling of the previous output of the DSM into the input sampling capacitor when the output of the DSM is reflected to the input terminal to boost the input impedance without the additional amplifier comprises: in an integration phase, integrating the previous output of the DSM into the input sampling capacitor, and integrating a charge difference between a current input voltage and the previous output into a feedback capacitor; in a pre-sampling phase, pre-sampling the previous output in the input sampling capacitor by an asynchronous SAR ADC; and in an input sampling phase, newly charging a next input voltage in the input sampling capacitor.

8. The method of claim 6, wherein the amplifying of the input signal using the inverter-based amplifier to obtain the wide input signal range comprises using a current-reuse inverter-based amplifier configured to increase a linear input range of the amplifier by not using a cascode stack for improved G_{M} low power consumption, and a wide bandwidth.

9. The method of claim 6, wherein the performing of the NS through the NS-SAR to reduce the truncation error, and then performing the truncation through the truncation logic to feed back the truncated output bits to the input DAC when the output bits of the DSM are reduced to the predetermined bits and fed back to and reflected in the input DAC comprises: reducing the output bits of the DSM to the predetermined bits and feeding the reduced bits back to the input DAC, thereby feeding back the truncation error and reusing the input DAC to perform the NS again with low power consumption.

10. The method of claim 9, wherein the performing of the NS through the NS-SAR to reduce the truncation error, and then performing the truncation through the truncation logic to feed back the truncated output bits to the input DAC when the output bits of the DSM are reduced to the predetermined bits and fed back to and reflected in the input DAC comprises: using a second-order NS-SAR, and additionally passing the truncation error fed back to the input DAC through the truncation logic through a second-order NS-SAR NTF so that second-order NS is additionally generated, thereby performing a total of fourth-order NS.
